(19) United States
(12) Patent Application Publication
(10) Pub. No.: US 2022/0104664 A1
(43) Pub. Date: Apr. 7, 2022

(54) SYSTEMS AND METHODS FOR SANITIZER DISPENSING DEVICES AND REMOTE MANAGEMENT OF THE SAME

(71) Applicant: UrgentRN LLC, San Juan, PR (US)

(72) Inventor: Brent M. Skoda, Mayfield Village, OH (US)

(21) Appl. No.: 17/323,661

(22) Filed: May 18, 2021

Related U.S. Application Data
(60) Provisional application No. 63/027,046, filed on May 19, 2020.

(51) Int. Cl. A47K 5/12; A61L 2/10; A61L 2/18; A61L 2/24; A61L 2/26

(52) U.S. Cl. CPC A47K 5/1217; A61L 2/10; A61L 2/18; A61L 2202/14; A61L 2/26; A61L 2202/121; A61L 2202/11; A61L 2/24

(57) ABSTRACT

According to one aspect, disclosed is a dispenser. The dispenser can include a housing. The housing can include a compartment for a container containing a substance for dispensing. The dispenser can include substance dispenser configured to dispense the substance from the container, the substance dispenser coupled to a substance-monitoring sensor. The dispenser can include a light emitter configured to emit light. The dispenser can include a communications module configured to communicate data between the dispensing system and a server. Processors can actuate the substance dispenser to dispense the substance responsive to a sanitation request. The processors can actuate the light emitter to emit light. The processors can determine, from the substance-monitoring sensor, an amount of substance remaining in the container. The processors can transmit, via the communications module, the amount to the server.

### CROSS-REFERENCE TO RELATED APPLICATION

This application claims priority to and the benefit of U.S. Patent Application No. 63/027,046, titled “SYSTEMS AND METHODS FOR SANITIZER DISPENSING DEVICES AND REMOTE MANAGEMENT OF THE SAME,” and filed on May 19, 2020, the contents of all of which are hereby incorporated herein by reference in its entirety for all purposes.

### FIELD OF THE DISCLOSURE

The present application relates generally to systems and methods for sanitizer dispensing, management and monitoring, and more particularly, to methods and systems for managing a sanitizer supply chain.

### BACKGROUND

Management of sanitation facilities requires either scheduling service visits to each facility or reducing the number of sanitation facilities. Oftentimes, sanitation workers servicing these sanitation facilities are unable to gauge the service need of the sanitation facilities they service and instead rely on scheduled service visits and sanitation complaints.

### SUMMARY

The present disclosure relates to systems and methods for a dispenser system and a multi-dispenser management system. According to one aspect, disclosed is a dispenser. The dispenser can include a housing. The housing can include a compartment for a container containing a substance for dispensing. The dispenser can include a substance dispenser configured to dispense the substance from the container. The substance dispenser can be coupled to a substance-monitoring sensor. The dispenser can include a light emitter configured to emit light. The dispenser can include an energy provider configured to provide energy to the substance dispenser and the light emitter. The dispenser can include a communications module configured to communicate data between the dispensing system and a server. The dispenser can include one or more processors. The processors can receive a request to initiate a sanitation process. The processors can actuate the substance dispenser to dispense the substance responsive to the request. The processors can actuate the light emitter to emit light. The processors can determine, from the substance-monitoring sensor, an amount of substance remaining in the container. The processors can transmit, via the communications module, the amount to the server.

In some implementations, the dispenser system can include a door coupled to the housing. The door can include a lock configured to restrict access to the container.

In some implementations, the dispenser system can include a user detector configured to detect a presence of a user, wherein receiving the request to initiate the sanitation request comprises generating the request responsive to the user detector detecting the presence of the user. In some implementations, the user detector identifies the user. Subsequent to the user detector identifying the user, the one or more processors can transmit, via the communications module to the server, usage data identifying the user.

In some implementations, the housing includes a mount configured to mount the housing to a wall surface or a stand.

In some implementations, the energy provider includes a magnetic levitation system configured to generate energy to control the dispenser. The magnetic levitation system includes a first magnet and a second magnet. The first magnet can exert a magnetic force on the second magnet responsive to the actuation of the substance dispenser to cause the magnetic levitation system to generate energy for a subsequent actuation of the substance dispenser.

In some implementations, the substance monitoring sensor is configured to determine the amount of the substance remaining in the container based on at least one of a weight of the substance remaining in the container, a visual marker indicating the amount of substance remaining in the container, or a force applied by the container to the substance monitoring sensor.

In some implementations, the dispenser system can include an actuator, the actuator actuated by the user to generate energy for the energy provider.

In some implementations, the energy provider includes at least one of a solar panel, a wind turbine, or a battery.

In another aspect, disclosed is a multi-dispenser management system. The multi-dispenser management system includes a communications manager configured to communicate with a plurality of dispensers. The multi-dispenser management system includes one or more processors coupled to memory. The processors can maintain, for each dispenser of the plurality of dispensers, in one or more data structures, a device profile including an amount of a substance remaining in the dispenser and location data of the dispenser. The processors can receive, from a dispenser, usage data corresponding to a usage event at the dispenser, the usage data identifying the dispenser. The processors can update, based on the usage data, the amount of substance remaining in the dispenser. The processors can determine that the amount of substance remaining in the dispenser satisfies a threshold corresponding to the dispenser. The processors can generate, responsive to determining that the amount of substance remaining in the dispenser satisfies the threshold, a refill request to refill the dispenser with the substance, the refill request identifying the dispenser. The processors can provide the refill request to an entity.

In some implementations, the processors identify from the usage data, a user corresponding to the usage data. The processors can maintain, in the one or more data structures, a user profile for the user including the usage data.

In some implementations, the usage data includes a quantity of substance dispensed by the dispenser. The processors can update the amount of substance remaining in the dispenser based on the quantity of substance dispensed.

In some implementations, the processors determine the threshold based on at least one of location data corresponding to a location of the dispenser or usage history data corresponding to previous usage events of the dispenser.

In some implementations, the processors determine a timestamp corresponding to the usage data received from the dispenser and determine the threshold based on the timestamp.

In some implementations, the processors can select, from a plurality of entities, the entity to which to provide the refill request. The processors can select the entity based on a current time or a location of the entity.

In some implementations, the usage data includes power data corresponding to a current power state of the dispenser. The processors can provide a notification to an entity based on the current power state.

In some implementations, the processors determine, for the dispenser, a first usage trend based on a first plurality of usage events received from the dispenser during a first time window and a second usage trend based on a second plurality of usage events received from the dispenser during a second time window. The processors can transmit a notification to an entity responsive to determining that the second usage trend is different from the first usage trend.

In some implementations, the processors can transmit, to a client device of the entity, door access data configured to unlock a door of the dispenser.

In some implementations, the processors can transmit, responsive to determining that the amount of substance remaining in the dispenser satisfies the threshold, one or more instructions to modify an amount of substance dispensed per usage event.

In some implementations, the processors can transmit, responsive to determining that the amount of substance remaining in the dispenser satisfies the threshold, one or more instructions to modify a length of time or an intensity of light emitted by the dispenser.

### BRIEF DESCRIPTION OF THE DRAWINGS

FIG. 1A is a block diagram depicting an embodiment of a network environment comprising local devices in communication with remote devices.

FIGS. 1B-1D are block diagrams depicting embodiments of computers useful in connection with the methods and systems described herein.

FIG. 2 is a block diagram depicting a dispenser configured to dispense products to a user.

FIG. 3 is a pictorial representation of the dispenser.

FIG. 4 is a block diagram depicting a multi-dispenser management system configured to manage the dispenser of FIGS. 2 and 3.

FIG. 5 is a table of an exemplary dispenser profile stored by the multi-dispenser management system for each dispenser.

FIG. 6 is a table of an exemplary user profile stored by the multi-dispenser management system for each user of the dispensers.

FIG. 7 is a flowchart diagram illustrating communications and processing flow involved in a method for managing a plurality of dispensers.

### DETAILED DESCRIPTION

For purposes of reading the description of the various embodiments below, the following descriptions of the sections of the specification and their respective contents may be helpful:

Section A describes a network environment and computing environment, which may be useful for practicing embodiments described herein.

Section B describes embodiments of systems and methods for sanitizer dispensing, and management and monitoring of dispenser.

Section C describes embodiments of systems and methods of remote dispenser management and monitoring.

### A. Computing and Network Environment

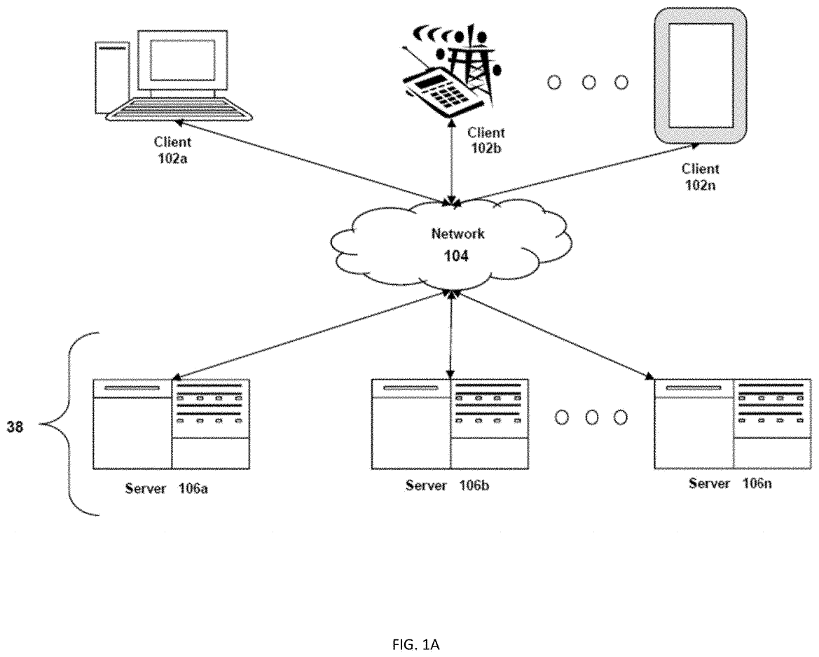

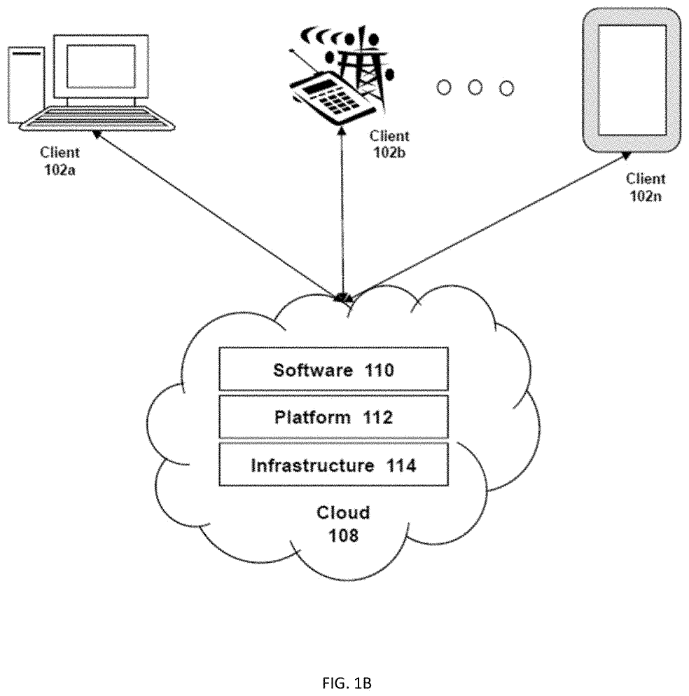

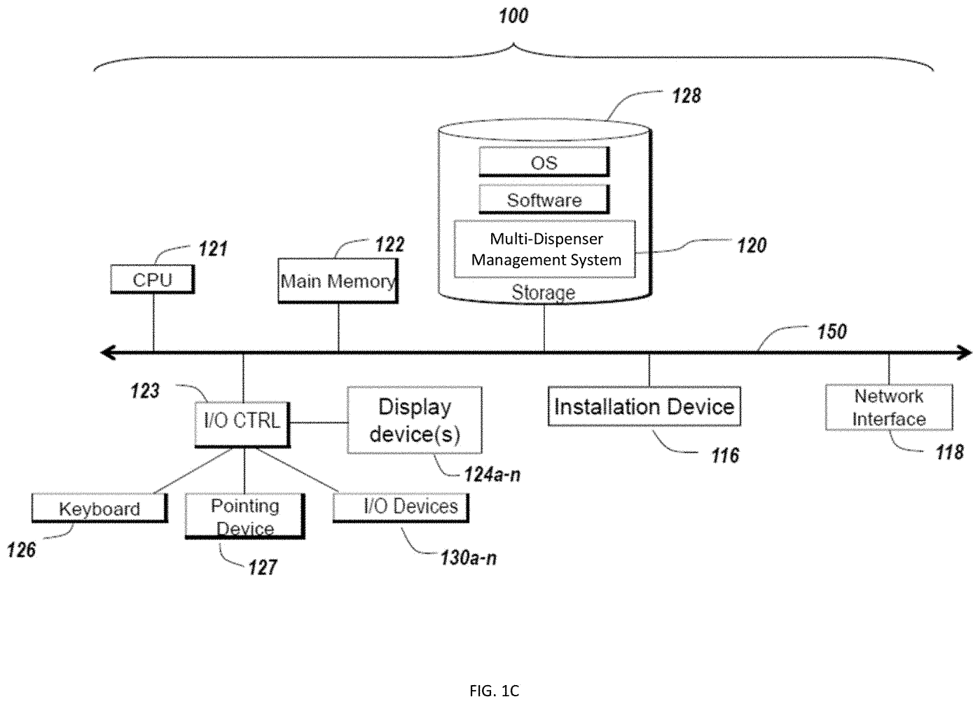

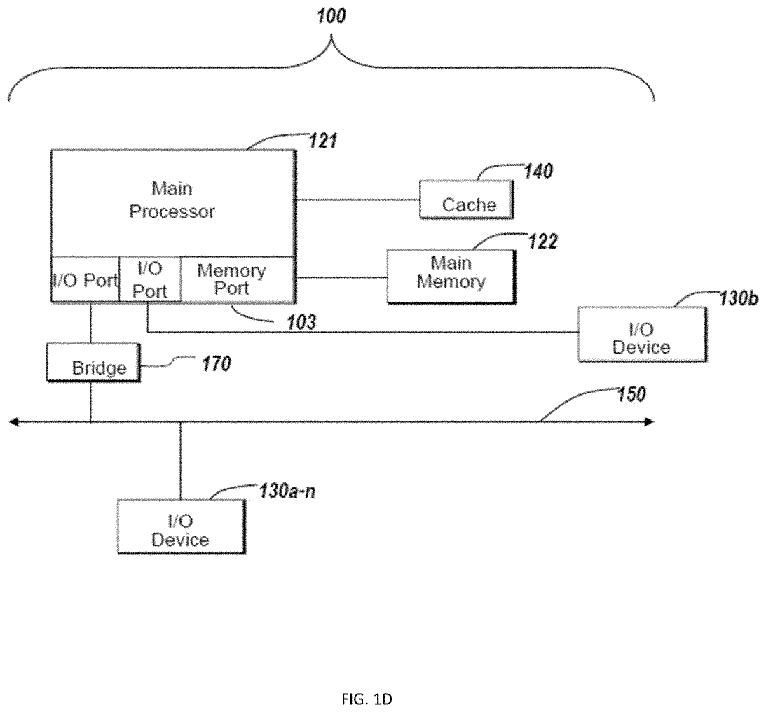

In addition to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 1G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms, or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS include AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 102 may also access SaaS resources through smartphone or tablet applications, including, for example, Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a multi-dispenser management system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of a multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software for the multi-dispenser management system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage device 128 may be external and connect to the computing device 100 via a I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some embodiments, the computing device 100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Wash. In other embodiments, the computing device 100 is an eBook reader, e.g. the KINDLE family of devices by Amazon.com, or NOOK family of devices by Barnes & Noble, Inc. of New York City, N.Y.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Systems and Methods for Sanitizer Dispensing, Management, and Monitoring

A smart sanitizer can provide sanitation services in a variety of locations without burdening service staff and supply chains. Since the smart sanitizer is efficient by relying on fewer resources, the availability of sanitation services can increase. The smart sanitizer can communicate with users over wireless communications technologies, so users can activate the smart sanitizer wirelessly. Users can activate the smart sanitizer to decontaminate surfaces. The smart sanitizer can decontaminate surface with a light and a sanitizing fluid. The smart sanitizer can track usage data of the smart sanitizer. The smart sanitizer can send the usage data to a management system. The smart sanitizer can adjust operation of the smart sanitizer based on the usage data. The usage data can include a request for service of the smart sanitizer or a refill of the sanitizing fluid.

According to at least one embodiment, the smart sanitizer can be a dispenser. The dispenser can include a housing. The housing can include a compartment for a container containing a substance for dispensing. The dispenser can include substance dispenser configured to dispense the substance from the container, the substance dispenser coupled to a substance-monitoring sensor. The dispenser can include a light emitter configured to emit light. The dispenser can include an energy provider configured to provide energy to the substance dispenser and the light emitter. The dispenser can include a communications module configured to communicate data between the dispensing system and a server. The dispenser can include one or more processors. The processors can receive a request to initiate a sanitation process. The processors can actuate the substance dispenser to dispense the substance responsive to the request. The processors can actuate the light emitter to emit light. The processors can determine, from the substance-monitoring sensor, an amount of substance remaining in the container. The processors can transmit, via the communications module, the amount to the server.

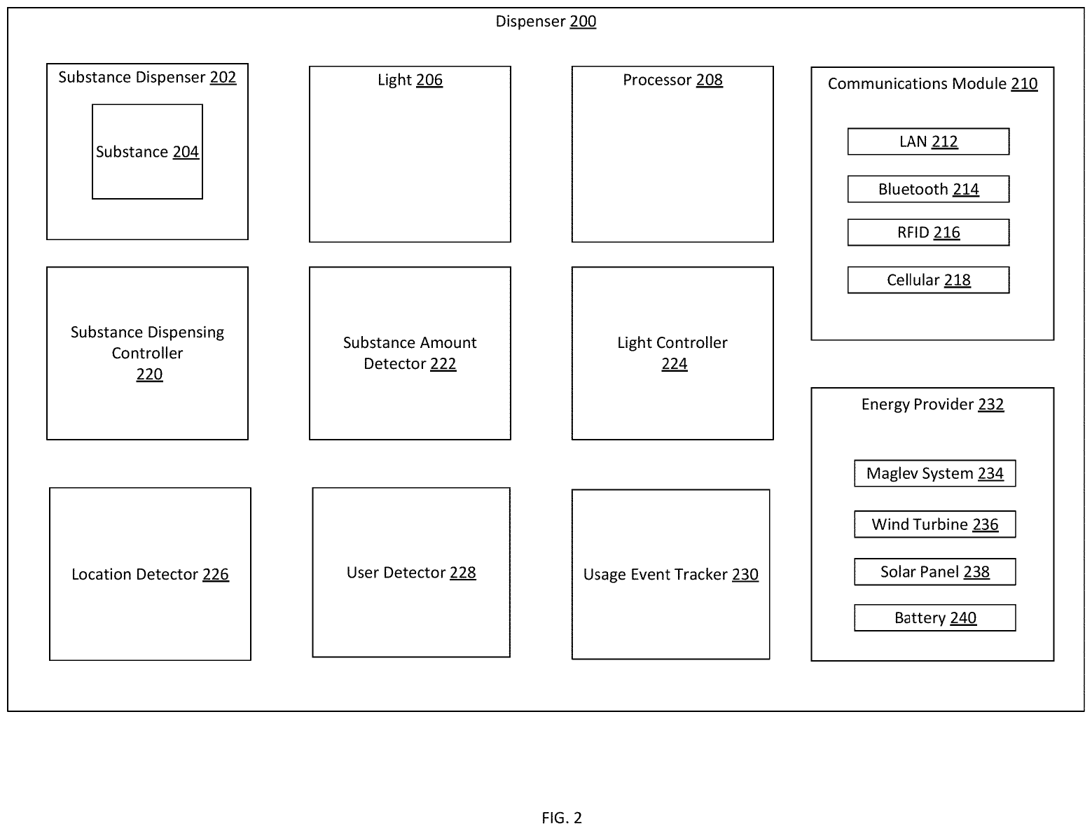

As shown in FIG. 2, depicted is a block diagram of the dispenser 200 for dispensing sanitizing solution. The dispenser can include a substance dispenser 202 configured to dispense a substance 204, such as a sanitizer solution. The dispenser 200 can also include a light 206 configured to emit light capable of disinfecting a surface, such as the skin of a user. A processor 208 executing instructions can operate the dispenser 200. The dispenser 200 can further include a communications module 210 for communicating with one or more remote devices. The communications module 210 can include at least one of a LAN connector 212, a Bluetooth connector 214, an RFID connector 216, or a cellular connector 218. The dispenser 200 can also include a substance-dispensing controller 220, a substance amount detector 222, a light controller 224, a location detector 226, a user detector 228, and a usage event tracker 230. The dispenser 200 can include an energy provider 232 configured to provide energy to various components of the dispenser 200. The energy provider 232 can obtain energy from one or more of a magnetic levitation system 234, wind energy convertor 236, solar energy convertor 238, or a battery 240.

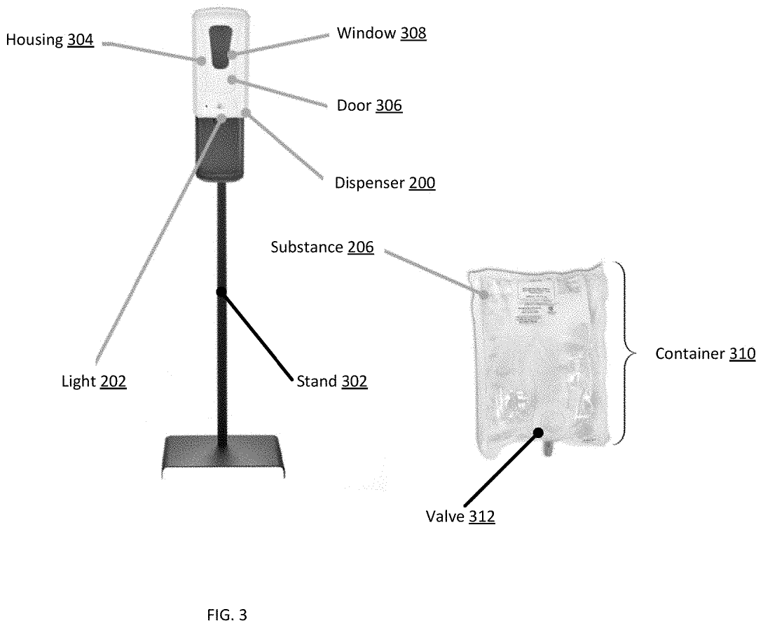

Referring now also to FIG. 3, FIG. 3 depicts a pictorial representation of the dispenser 200. A stand 302 can support the dispenser 200. The dispenser 200 can includes a housing 304, which can include a door 306 to access the substance dispenser 202 and a window 308. As shown in FIG. 3, a container 310 can include the substance 204 and a valve 312 for controlling the dispensing of the substance 204 from the container 310. The container 310 can be disposed within the substance dispenser 202 of the dispenser 200.

The size and shape of the housing 304 of the dispenser 200 can allow for the inclusion of the components of the dispenser shown in FIG. 2. The size and shape of the housing 304 can allow for mounting the dispenser 200 to a wall. The size and shape of the housing 304 can allow stand, such as the stand 302 to support the dispenser 200. The housing 304 can include the door 306 to access the substance dispenser 202. The position of the door 306 can be towards a front or a back of the dispenser 200. The door 306 can include a lock to restrict access to the compartment. The dispenser can allow the lock to be remotely controlled. The position of the window 308 can be adjacent to or on the door 306 and can allow a user to view the inside of the substance dispenser 202 without having to open the door 306. For instance, a user can view the amount of substance remaining in the container and/or substance dispenser 202 via the window 308.

The housing 304 defines the substance dispenser 202. The substance dispenser 202 can store the substance 204. The substance 204 can be stored in a container 310. The position or installation of the container 310 can be in the substance dispenser 202. The substance dispenser 202 can include an actuator. The dispenser 200 can control the actuator. The actuator can to dispense the substance. The substance dispenser 202 can also include one or more sensors for determining an amount of substance 204 dispensed. The substance dispenser 202 can also include one or more sensors for determining an amount of substance 204 remaining.

The substance dispenser 202 can store the container 310 or a plurality of containers. The container 310 can contain the substance 204. The substance dispenser 202 can store the substance 204 or the container 310 having the substance 204. The container 310 can be a pouch. The sensors of the substance dispenser 202 can determine the quantity and type of substance 204 in the container 310. The container 310 can include a valve 312. In some embodiments, the valve 312 is rubber or mechanical. The actuator controlled by the substance-dispensing controller 220 can actuate the valve 312 to regulate the speed and quantity of substance 204 flowing from the dispenser 200.

Referring now to FIGS. 2 and 3, the substance dispenser 202 can dispense a substance 204. The substance dispenser 202 can receive a container including the substance 204. In some embodiments, the substance 204 is hand sanitizer, rubbing alcohol, or wipes. The substance dispenser 202 can include an actuator to actuate a valve to dispense the substance 204 from the container.

In some embodiments, the actuator can actuate the valve by twisting, squeezing, or opening the valve. Actuating the valve can dispense the substance 204. Specific actuations of the valve can control the amount of substance dispensed. For instance, the actuator can actuate the valve for various lengths of time to dispense different amounts of substance 204. The substance dispenser 202 can be controlled by the processor 208 or the substance dispensing controller 220 executed by the processor 208. The substance-dispensing controller 220 describes additional details regarding controlling the dispensing of the substance 204.

The light 206 of the device can emit energy to decontaminate or disinfect a surface, such as by eliminating viruses like coronavirus. The light 206 can be positioned inside or on the outside the dispenser 200. The light 206 can include one or more UV lamps, and the dispenser 200 can include a plurality of lights. The light 206 can be an add-on attachment to the dispenser 200. The light 206 can emit UV light. The light 206 can emit light adjacent to where the substance 204 exits the dispenser 200. The light 206 can emit the UV light in the same direction as the direction of the substance 204 exiting the dispenser 200. The direction of the UV light can be adjustable. The light 206 can be controlled by the processor 208 or the light controller 224 executed by the processor 208. For instance, the duration, brightness, direction, or intensity of the light 206 can be controllable.

The substance-dispensing controller 220 can include hardware, software or a combination of hardware and software. The substance dispensing controller 220 can include a script, program, file, or other software construct, which when executed by the processor 208, can be configured to control the substance dispenser 202 to dispense the substance 204. The substance-dispensing controller 220 can cause the substance dispenser 202 to dispense the substance 204 responsive to receiving a command. In some embodiments, the substance-dispensing controller 220 receives the command responsive to the user detector 228 detecting the presence of a user. The user detector 228 describes additional details regarding detecting users.

The substance-dispensing controller 220 can send a control signal to the substance dispenser 202 to release the substance 204. The control signal can actuate the actuator of the substance dispenser 202. The control signal can control the rate at which the substance dispenser dispenses the substance. For instance, the control signal can cause the actuator of the substance dispenser 202 to open the valve for a predetermined amount of time. In some embodiments, the control signal can control the actuator to alter the size of an opening of a valve to control an amount of substance dispensed per unit time while the valve remains open. The control signal to control the amount of substance 204 dispensed can be based on one or more factors, including the user, type of user, a time of day, a location of the dispenser 200, and the amount of substance 204 remaining in the device, amount of power or energy available to the dispenser 200, among others.

The substance amount detector 222 can include hardware, software or a combination of hardware and software. The substance amount detector 222 can include a script, program, file, or other software construct, which when executed by the processor 208, can be configured to detect the amount of substance 204 remaining in the substance dispenser 202. In some implementations, the substance amount detector 222 determines the amount of the substance 204 remaining in the dispenser 200 or the substance dispenser 202. The substance amount detector 222 can detect the amount of substance 204 based on at least one of a weight of the substance 204 remaining in the substance dispenser. The substance amount detector 222 can determine the amount of substance 204 dispensed by the substance dispenser 202 to determine the amount of substance 204 remaining. The substance amount detector 222 can identify a total capacity of the container of the substance 204 and based on the amount of substance dispensed from the container, the substance amount detector can calculate the amount of substance remaining by subtracting the amount of substance dispensed from the container from the total capacity of the container. The substance amount detector 222 can communicate with or include an optical sensor or other type of sensor configured to detect a marker indicating the amount of substance 204 remaining in the substance dispenser 202. In some embodiments, the substance amount detector 222 can communicate with or include a force sensor configured to determine or measure the amount of substance 204 remaining in the substance dispenser 202. The force sensor can determine a force applied by the substance 204 to the force sensor. In some embodiments, the one or more sensors in communication with the substance amount detector determine the amount of substance remaining in the container or the dispenser. A strategic position or arrangement within the device can allow the one or more sensors to generate measurements of a volume, depth, or weight of the remaining amount of substance 204 in the dispenser 200. The substance amount detector 222 can determine the remaining amount of substance 204 based on the measurements.

The substance amount detector 222 can include one or more sensors for sensing or monitoring the amount of substance dispensed from or remaining in each container 310. In some implementations, the sensors can monitor a weight of the substance 204 dispensed or a weight of substance 204 remaining in the container 310 after substance is dispensed. In some implementations, the sensors can count a number of containers of substance 204 dispensed. In some implementations, the sensors can monitor a time for which a valve 312 is open and a flow rate of the substance flowing through the valve 312. In some implementations, the substance amount detector 222 can determine an amount of substance 204 remaining in the dispenser 200 based on the amount of substance 204 originally included in a container 310 and an amount of substance 204 already dispensed since the container 310 was first inserted. The substance amount detector 222 can be configured to communicate quantity levels of the substance 204 to the usage event tracker 230 such that the multi-dispenser management system 120 can submit a refill request for substance 204 or container 310 refills to one or more service staff before the dispenser 200 runs out of substance 204.

The light controller 224 can include hardware, software or a combination of hardware and software. The light controller 224 can include a script, program, file, or other software construct, which when executed by the processor 208, can control or regulate the operation of the light 206. The light controller 224 can control the light 206 by sending a control signal to the light 206. The control signal can activate the light 206. The control signal can cause the light 206 to emit light in intervals or according to one or more light patterns. The control signal can control a length of time that the light 206 is on or activated. The control signal can control the intensity of light 206. The control signal can set a time delay between a request to turn the light on and the light 206 turning on. The control signal can include on one or more factors such as the user, type of user, time of day, location of the dispenser 200, and the amount of substance 204 remaining in the device. The light controller 224 can transmit data indicating the actions of the light controller to the usage event tracker 230.

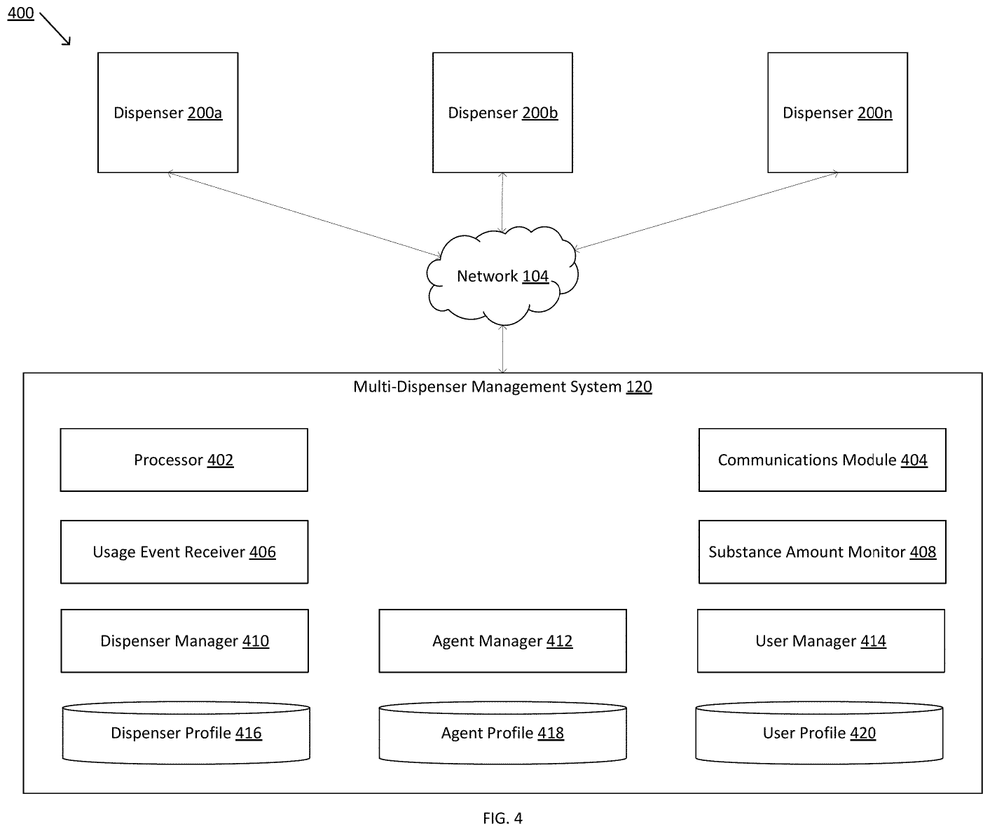

The location detector 226 can include hardware, software or a combination of hardware and software. The location detector 226 can include a script, program, file, or other software construct, which when executed by the processor 208, can determine the location of the dispenser 200. The location detector 226 can determine a location of the dispenser 200. The location detector 226 can communicate with or include a global positioning system (GPS) to determine the location of the dispenser 200. The location can include the positioning coordinates of the dispenser 200. In some embodiments, the location detector 226 can identify a location of the dispenser 200 within a building or an enclosed area. The location detector 226 can determine if the dispenser 200 is moving. The location detector 226 can determine if the current location of the dispenser 200 is different from a preset location of the dispenser 200. For instance, a difference between the current location and the preset location can indicate the dispenser 200 is stolen or in service. The location detector 226 can transmit location data to the substance-dispensing controller 220, which the substance-dispensing controller 220 can use to control an amount of substance 204 dispensed. The location detector 226 can transmit location data to the light controller 224, which the light controller 224 can use to determine a light intensity or duration. For instance, the light controller 224 can control the light 206 differently based on whether the location data indicates that the dispenser is indoors or outdoors. The location detector 226 can transmit location data to the multi-dispenser management system 120 to arrange refill requests. For instance, the location detector 226 can transmit the location data via the communications module 210 to the multi-dispenser management system 120 to indicate where the service staff should service the dispenser 200 and refill the substance 204. FIGS. 4, 5, and 6 depict additional information about the data and multi-dispenser management system.

The user detector 228 can include hardware, software or a combination of hardware and software. The user detector 228 can include a script, program, file, or other software construct, which when executed by the processor 208, can detect a presence of a user of the dispenser 200. In some implementations, the dispenser 200 can include a user detector 228 configured to detect a presence of a user. The user detector 228 can detect motion associated with the user. The user detector 228 can detect a device associated with the user. In some embodiments, the user detector 228 can detect the device associated with the user based on one or more communication signals emitted by the dispenser 200 or the device, including BLUETOOTH or other short-range communication signals. The device can be a communication device, such as a mobile device, portable device, wearable device, or computer device. Based on the device associated with the user, the user detector 228 can identify the user and their user data such as a user identifier, user event identifier, user name, and user start time. In some implementations, the user detector 228 identifies the user. The user detector 228 can include or communicate with one or more sensors to determine or measure vitals or physiological metrics of a user, such as their temperature or weight. The vitals or physiological measurements can include body temperature measurement, blood pressure measurement, pulse measurement, blood sugar measurement, blood-oxygen level measurement, heart beat measurement, or a combination thereof. For instance, the user detector 228 can communicate with sensors or sensing modules for performing such measurements, or the dispenser 200 can include wearable or portable measuring devices communicatively coupled with the dispenser 200. In some implementations, the user detector 228 transmits usage data to the multi-dispenser management system 120. The usage data can identify the user subsequent to the user detector 228 identifying the user. The user detector 228 can transmit the usage data to the multi-dispenser management system 120 via the communications module 210. Responsive to the user detector 228 identifying the user, the user detector 228 can transmit, via the communications module 210, the usage data to the multi-dispenser management system 120. FIGS. 4, 5, and 6 depict additional information about the data and multi-dispenser management system.

Responsive to detecting the presence of a user, the user detector 228 can transmit or provide the user data to the substance-dispensing controller 220 and the light controller 224. In some implementations, the user detector 228 can generate a request to initiate a sanitation request responsive to the user detector 228 detecting the presence of a user. In some embodiments, the user detector 228 can determine the identity of the user. Based on the identity of the user, the user detector 228 can access user profile data of the user to enable the dispenser 200 (or its components) to use the user profile data to customize the operation of the dispenser 200 based on the user's preferences.

The user detector 228 can also detect a dispensing request to dispense the substance 204. The dispensing request can include user motions, touch inputs, voice inputs, or visual inputs. The user detector 228 can detect or receive user motions, touch inputs, voice inputs, or visual inputs via one or more user interface components or sensors, cameras, microphones, among others. The dispensing request can include a dispensing request signal from the user's device, or the application of a dispensing input component. The dispensing input component can include a button, a lever or a pressure-based mat disposed on or adjacent to the dispenser 200. The dispensing input component can include a sensor that detects the dispensing request. Responsive to detecting the dispensing request via the dispensing input component, the user detector 228 can transmit a control signal to the substance-dispensing controller 220 to initiate dispensing of substance 204. In some embodiments, responsive to detecting the dispensing request, the user detector 228 can transmit a control signal to the light controller 224 to initiate use of the light 206. In some embodiments, the substance dispensing controller 220 can dispense the substance responsive to an action performed on or via the dispensing input component and the light controller 224 can activate the light 206 responsive to an action performed on or via the dispensing input component.

The usage event tracker 230 can include hardware, software or a combination of hardware and software. The usage event tracker 230 can include a script, program, file, or other software construct, which when executed by the processor 208, can track the operation of the dispenser 200. The usage event tracker 230 can count the number of times the substance dispenser 202 dispensed the substance 204. The usage event tracker 230 can count the number of times the substance dispenser 202 actuated the valve to dispense the substance 204. The usage event tracker 230 can track the operation of the substance-dispensing controller 220 to determine the frequency of substance dispensing, or the amount (volume or mass) of the substance dispensed each time. The indication of a substance 204 dispensing event can include information indicative of occurrence of a dispensing event, such as one-bit signal, information indicative of an amount of the substance 204 that was dispensed, time of dispensing event, location of dispensing event, or a combination thereof. The usage event tracker 230 can track measurements of the substance amount detector 222 to determine how much of the substance 204 is remaining in the substance dispenser 202 at any given time. The measurements can include an amount of substance 204 dispensed or a level of the substance 204 remaining in one or more substance containers.

The usage event tracker 230 can track operation of the light controller 224 to determine when the light 206 activates. The usage event tracker 230 can determine, for each time the light is activated, one or more characteristics of the light, including the intensity of the light, the length of time the light is activated, and one or more light patterns according to which the light is activated. The usage event tracker 230 can generate usage data of the light controller 224.

The usage event tracker 230, via the processor 208, can maintain a log storing information related to the monitoring of the dispenser 200. The log can store information related to communications received from the multi-dispenser management system 120. The log can store event related information. An exemplary event is the substance-dispensing controller 220 controlling the substance dispenser 202 to dispense the substance 204. Exemplary events can further include the light controller 224 controlling the light 206, a user or multi-dispenser management system 120 establishing communications with the dispenser 200, an alarm triggering event (in the event of an attempt to tamper the dispenser 200), location changes of the dispenser 200, or changes to ambient conditions of the dispenser 200. For instance, when the dispenser 200 senses a temperature below or above one or more predefined temperatures, detecting low levels of the substance 204 in each substance dispenser 202 of the dispenser 200, among others. The log can identify a date and time at which substance 204 was dispensed, an amount of substance 204 dispensed as well as any other measurements, For instance, temperature, pulse, flow rate, substance viscosity, substance levels, among others, taken around the time the substance was dispensed. In addition to substance dispensing related activity, the log can identify when a user or service staff member communicated or interacted with the dispenser 200. In some implementations, the log can include information related to changes of the amount of substance 204 dispensed by the substance dispensing controller 220 as well as changes in the time at which the substance 204 is to be administered.

The usage event tracker 230 can send measurement data and/or other information to the multi-dispenser management system 120. The usage event tracker 230 can transmit the data via the communications module 210 to the multi-dispenser management system 120. FIGS. 4, 5, and 6 depict additional information about the data and multi-dispenser management. In some implementations, the usage event tracker 230 reports each dispensing event to the multi-dispenser management system 120. The multi-dispenser management system 120 can log information indicative of dispensing events, such as dispensing event timing, dispensing event location, amount of the substance dispensed, or a combination thereof. For instance, the usage event tracker 230 can store such information in memory. For instance, the usage event tracker 230 can send, to the multi-dispenser management system 120, dispenser 200 related information. The dispenser 200 related information can include an indication of an attempt to tamper the dispenser 200, indication of a substance 204 dispensing event, indication of ambient condition(s) associated with the dispenser 200, indication of substance 204 expiration, or a combination thereof. The indication of an attempt to tamper the dispenser 200 can be in the form of one-bit signal or can include information indicative of a type of tampering, time of tampering attempt, result of tampering attempt or a combination thereof. The information sent by the usage event tracker 230 can also include an identification of the dispenser 200. The usage event tracker 230 can transmit the data via the communications module 210 to the multi-dispenser management system 120. FIGS. 4, 5, and 6 depict additional information about the data and multi-dispenser management system.

An energy provider 232 can provide energy for the operation of the dispenser 200. The energy provider 232 can track the amount of energy generated or provided to the dispenser 200. The energy provider 232 can transmit, to the usage event tracker 230, the amount of energy generated and energy provided. In some implementations, the energy provider 232 includes at least one of a magnetic levitation system 234, a wind energy convertor 236, a solar energy convertor 238, or a battery 240.

A magnetic levitation system 234 can generate energy for the energy provider 232 from magnetic driven electricity generated by the operation of the dispenser 200. The magnetic levitation system 234 can include a first magnet and a second magnet. The first magnet can exert a magnetic force on the second magnet, which causes the magnetic levitation system 234 to generate energy for the dispenser 200. In some implementations, the dispenser 200 can include an actuator coupled to the substance dispenser 202 or the dispenser 200. The user can generate energy for the dispenser 200 by actuating the actuator. The actuator can cause the first magnet of the magnetic levitation system 234 to exert a magnetic force on the second magnet responsive to the actuation of the actuator, which causes the magnetic levitation system 234 to generate energy for a subsequent actuation of the actuator. In some implementations, the first magnet is coupled to the actuator. The first magnet can generate a magnetic field. Kinetic energy can transfer from the actuator to the first magnet, which causes movement of the first magnet, and induces an electrical current in an electromagnetic coil, causing electricity generation. The magnetic levitation system 234 can store the generated electricity in the battery 240 or other energy storage device, or output the generated electricity via the energy provider 232 to the dispenser 200.

The wind energy convertor 236 can generate energy for the energy provider 232 from wind. In some embodiments, the wind energy convertor 236 can couple to the dispenser 200 or the energy provider 232.

The solar energy convertor 238 can generate energy for the energy provider 232 from solar energy. The solar panel can generate energy from natural sunlight or ambient light. In some embodiments, the solar energy convertor 238 can couple to the dispenser 200 or affix to the housing or stand of the dispenser 200.

The battery 240 can store energy for the energy provider 232. The battery 240 can be rechargeable or replaceable by service staff. The magnetic levitation system 234, the wind energy convertor 236, or the solar energy convertor 238 can charge the battery 240.

The communications module 210 can include hardware, software or a combination of hardware and software. The communications module 210 can include a script, program, file, or other software construct, which when executed by the processor 208, can be configured to communicate with one or more users and one or more multi-dispenser management systems 120. The communications module 210 can include a unique communications identifier for the dispenser 200. The communications module 210 can include one or more adapters, ports, or connectors for communication. The communications module 210 can communicate over the LAN connector 212, the Bluetooth connector 214, the RFID connector 216, or the cellular connector 218. In some embodiments, other communications technologies and protocols can allow for the communication between the user and the multi-dispenser management system 120. The LAN connector 212 of the communications module 210 can communicate with the users and the multi-dispenser management system 120 via local area networking technologies. The Bluetooth connector 214 of the communications module 210 can communicate with the users and the multi-dispenser management system 120 via wireless technology standards utilizing short wavelength radio waves over short distances. The RFID connector 216 of the communications module 210 can obtain digital data from the users via wireless technology standards. The cellular connector 218 of the communications module 210 can communicate with the users and the multi-dispenser management system 120 via wireless networking technologies.

### C. Systems and Methods of Remote Sanitizer Management and Monitoring

A multi-dispenser management system can remotely communicate with one or more dispenser devices, such as the dispenser 200 shown in FIG. 2. The multi-dispenser management system can monitor the status of each of the dispenser devices, including monitoring the usage of the dispenser devices. The multi-dispenser management system can monitor an amount of substance each dispenser device has dispensed as well as the amount of substance remaining in each dispenser device. The multi-dispenser management system can analyze the usage data as well as monitor the amount of substance in each dispensing device for automatically generating refill requests or other service requests to service the dispensing devices. In this way, the multi-dispenser management system can manage the servicing of dispensers by efficiently directing service agent efforts to relevant dispensers and optimizing the availability of supplies for a plurality of dispensers. Since the multi-dispenser management system can optimize the usage of service agent and supply resources, a team of service agents can deploy and service a greater number of dispensers to increase the availability of sanitation services. The multi-dispenser management system can communicate with service agents and the dispensers over wireless communications technologies. For instance, the multi-dispenser management system can wirelessly receive data from a dispenser device. The multi-dispenser management system can determine, from the data, that the dispenser device needs service. The multi-dispenser management system can generate and transmit a service request to an agent who can process the service request and service the dispenser. The multi-dispenser management system can manage usage data of each dispenser, service data pertaining to the service agents that service the dispensers, and user data for users that use the dispensers. For instance, the multi-dispenser management system can track the locations of the dispensers and the service agents, and monitor the usage of the dispensers by the users. The multi-dispenser management system can regulate the availability of the dispensers. For instance, the multi-dispenser management system can turn off dispensers or restrict certain users from using the dispensers.

In some embodiments, the multi-dispenser management system includes a communications manager configured to communicate with a plurality of dispensers. The multi-dispenser management system includes one or more processors coupled to memory. The processors can maintain, for each dispenser of the plurality of dispensers, in one or more data structures, a device profile including an amount of a substance remaining in the dispenser and location data of the dispenser. The processors can receive, from a dispenser, usage data corresponding to a usage event at the dispenser, the usage data identifying the dispenser. The processors can update, based on the usage data, the amount of substance remaining in the dispenser. The processors can determine that the amount of substance remaining in the dispenser satisfies a threshold corresponding to the dispenser. The processors can generate, responsive to determining that the amount of substance remaining in the dispenser satisfies the threshold, a refill request to refill the dispenser with the substance, the refill request identifying the dispenser. The processors can provide the refill request to an entity.

As shown in FIG. 4, depicted is an environment 400 of that includes the multi-dispenser management system 120 configured to manage one or more dispensers 200a-200n via a network 104.

The multi-dispenser management system 120 can include a processor 402 (similar to processor 121 described in Section A) and a communications module 404 for enabling the multi-dispenser management system 120 to communicate with one or more dispensers or agents. The communications module 404 can include at least one of a LAN connector, a Bluetooth connector, an RFID connector, or a cellular connector. The multi-dispenser management system 120 can include a usage event receiver 406 configured to receive usage event data from each dispenser 200, such as a substance dispensing event. The multi-dispenser management system 120 can also include a substance amount monitor 408 configured to monitor the amount of substance 204 in each dispenser 200.

The multi-dispenser management system 120 can also include a dispenser manager 410 configured to manage each dispenser 200. The multi-dispenser management system 120 can also include an agent manager 412 configured to manage each agent servicing the dispensers. The multi-dispenser management system 120 can also include a user manager 414 configured to manage each user using the dispensers.

The multi-dispenser management system 120 can include a dispenser profile 416 configured to store data associated with each dispenser 200. The multi-dispenser management system 120 can also include an agent profile 418 configured to store data associated with each agent servicing the dispensers. The multi-dispenser management system 120 can also include a user profile 426 configured to store data associated with each user using the dispensers.

The communications module 404 can allow the multi-dispenser management system 120 to communicate with the dispensers 200a-200n. The communications module 404 can transmit and receive data packets to and from the dispensers via one or more communication channels established via the one or more networks 104. The communications module 404 can establish communications with the agents. The agents can be service staff that service the dispensers. In some embodiments, the communications module 404 can enable the multi-dispenser management system 120 to communicate with one or more third-party data sources. For instance, the multi-dispenser management system 120 can access one or more weather data sources, location data sources, among others.

The communications module 404 can communicate with the dispensers or devices of one or more service agents via at least one of a LAN connector or port, a Bluetooth connector or port, an RFID connector or port, or a cellular connector or port, among other communication ports.

The usage event receiver 406 of the multi-dispenser management system 120 can include hardware, software or a combination of hardware and software. The usage event receiver 406 can include a script, program, file, or other software construct, which when executed by the processor 402, can receive data corresponding to one or more events occurring at the dispenser 200. The events can include user events such as detecting a user, dispensing substance, servicing the dispensers, or actuating the light. In some embodiments, the user events includes power data corresponding to power states of the dispenser. The events can also include agent events such the agent servicing the dispenser 200, moving the dispenser 200, refilling the dispenser 200 with substance 204. The data can be can include packets or information such as the amount of substance dispensed or the actuation time of the light.

Responsive to a dispensing event occurring at a dispenser, the usage event receiver 406 can receive dispensing event data corresponding to the dispensing event from the dispenser 200. The dispensing event data can include a dispenser identifier identifying the dispenser at which the dispensing event occurred. The dispensing event data can include a dispenser location identifying a location of the dispenser. The dispensing event data can include an event identifier identifying the event, an event type indicating that the event was a dispensing event. The dispensing event data can include a user identifier identifying a user or agent associated with the event, an amount of substance dispensed, an amount of substance remaining in the substance, and a timestamp corresponding to when the event occurred. In addition, the dispensing event data can include data corresponding to the light of the dispensing device, including a length of time the light was actuated, and one or more characteristics of the light emitted, including a light color, light intensity, light pattern, among others.

Responsive to a service event occurring at the dispenser, the usage event receiver 406 can receive service event data from either the dispenser 200 or a device of the agent servicing the dispenser. The service event data can include data corresponding to the service. The service event data corresponding to the service can include a dispenser identifier identifying the dispenser at which the service event occurred. The service event data corresponding to the service can include a dispenser location identifying a location of the dispenser. The service event data corresponding to the service can include an event identifier identifying the event. The service event data corresponding to the service can include an event type indicating that the event was a service event. The service event data corresponding to the service can include an agent identifier identifying the agent performing the service. The service event data corresponding to the service can include a timestamp corresponding to a performance or completion of the service. In addition, the service event data can include additional details indicating service of the light 206, refilling or replacing of the substance 204, among others. In some embodiments, the service event data can further include a current amount of substance in the dispensing device.

The substance amount monitor 408 can include hardware, software or a combination of hardware and software. The substance amount monitor 408 can include a script, program, file, or other software construct, which when executed by the processor 402, can monitor the amount of substance 204 in the dispenser 200. In some embodiments, the substance amount monitor 408 determines, from the device profile corresponding to the device, an amount of substance remaining in the device. Provided below are details regarding updating the device profile. In some embodiments, the substance amount monitor 408 can identify an amount of substance remaining in a dispenser based on the dispensing event data received by the usage event receiver 406 from the dispenser. In some embodiments, the substance amount monitor 408 can identify an amount of substance remaining in a dispenser from a value stored in the dispenser profile of the device. In some embodiments, the substance amount monitor 408 can identify an amount of substance remaining in the dispenser by subtracting the amount of substance dispensed during a dispensing event from the value stored in the dispenser profile corresponding to the amount of substance remaining in the dispenser.

The dispenser manager 410 can include hardware, software or a combination of hardware and software. The dispenser manager 410 can include a script, program, file, or other software construct, which when executed by the processor 402, can perform various functions relating to the management of one or more of the dispensers. The dispenser manager 410 can maintain dispenser profiles for one or more dispensers. The dispenser manager 410 can maintain, in one or more data structures, dispenser profiles for the dispensers. The dispenser manager 410 can periodically update a dispenser profile of a dispenser. In some embodiments, the dispenser manager 410 can update the dispenser profile of a dispenser responsive to a dispensing event or service event performed at the dispenser or the usage event receiver 406 receiving data packets corresponding to a dispensing event or a service event.

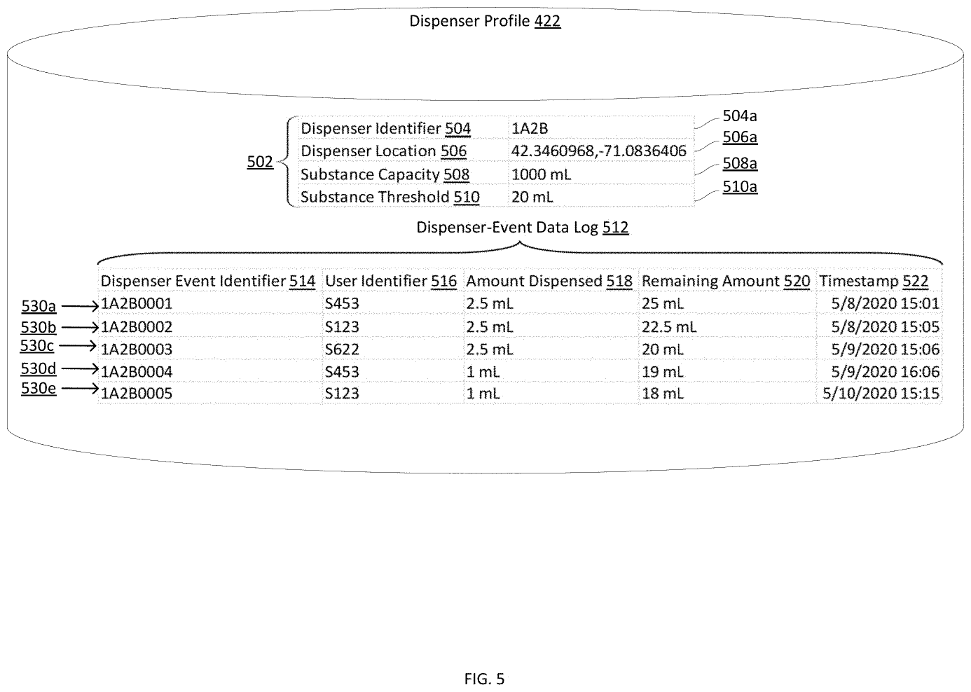

Referring briefly also to FIG. 5, FIG. 5 shows a block diagram of a representative dispenser profile of a dispenser. The dispenser profile 416 can include specification dispenser data 502. The specification dispenser data 502 can be preset or static data corresponding to the dispenser. The specification dispenser data 502 can include a dispenser identifier 504 identifying the dispenser 200. The specification dispenser data 502 can include a dispenser location 506 identifying the location of the dispenser 200. The specification dispenser data 502 can include a substance capacity 508 indicating the amount of substance 204 that the dispenser 200 can hold.

The specification dispenser data 502 can include a substance threshold 510 indicating the amount of substance 204 that may trigger one or more actions pertaining to the dispenser 200. The dispenser profile 416 can include a dispenser-event data log 512. The dispenser-event data log 512 can include one or more dispenser entries 530a-e. Each dispenser entry 530 can include a dispenser event identifier 514 identifying each event of the dispenser 200. Each dispenser entry 530 can include a user identifier 516 identifying a user using the dispenser 200 during the dispensing event. Each dispenser entry 530 can include an amount dispensed 518 indicating the amount of substance 204 dispensed during the dispensing event. Each dispenser entry 530 can include a remaining amount 520 indicating the amount of substance 204 left in the substance dispenser 202 or container 310 after the dispensing event. Each dispenser entry 530 can include and a timestamp 522 indicating a time during which each event occurred. In some embodiments, the dispenser profile 416 can receive and store additional data attributes from the dispenser manager 410. The data can be in hexadecimal, numerical, or any other database format.

In an exemplary embodiment, the dispenser identifier 504a of the dispenser 200 is “1A2B.” Dispenser location 506a is in coordinates that indicate the location of the dispenser. The dispenser manager 410 can update the coordinates with the updated location of the dispenser 200. The dispenser profile 422 can store previous locations of the dispenser 200. Substance capacity 508a is “1000 ml”, and substance threshold 510a is “20 ml”. Each dispenser entry 530 stored in the dispenser event data log 512 can correspond to a dispensing event or a service event performed at the dispenser. Each dispenser entry 530 can include a dispenser event identifier uniquely identifying the event. Each dispenser entry 530 can include a user identifier identifying the user. Each dispenser entry 530 can include an amount of substance dispensed. Each dispenser entry 530 can include a remaining amount of substance. Each dispenser entry 530 can include a timestamp indicating an occurrence time of the event. The user identifier indicates the specific user that interacted with the dispenser either as a user or as an agent. The amount dispensed indicates the amount of substance dispensed. The remaining amount indicates how much of the substance 204 remained in the substance dispenser 202 after the dispensing event. The timestamp indicates when the user interaction with the dispenser 200 took place. Each dispenser event identifier 514 can also correspond to light settings and light usage of the dispenser 200. For instance, the dispenser event identifier 514 can correspond to a data structure storing that the light was on during the dispensing event, or any other parameter transmitted by the light controller 224 of the dispenser 200 and received by the usage event receiver 406 for analysis by the dispenser manager 410.

The dispenser manager 410 can update one or more parameters indicative of substance amount dispensed, substance remaining in the dispenser 200 or a container 310 therein, number of dispensing events, or a combination thereof. For instance, based on the quantity of substance dispensed, the dispenser manager 410 can update various values of the device profile, including but not limited to the amount of substance remaining in the dispenser 200.

The dispenser manager 410 can compare one or more updated parameters to one or more respective threshold values to determine whether the dispenser 200 needs service or whether the dispenser needs a substance refill. In some embodiments, the dispenser manager 410 determines a timestamp corresponding to the usage data received by the usage event receiver 406 from the dispenser 200, and determine the threshold based on the timestamp. For instance, if the amount of substance remaining in the substance dispenser 202 or a container 310 is below a respective threshold (or the amount of substance 204 dispensed since a given date or event is above another respective threshold), the dispenser manager 410 can determine that a refill is needed before the dispenser 200 runs out of substance 204. In some embodiments, the dispenser manager 410 transmits, responsive to determining that the amount of the substance remaining in the dispenser 200 satisfies the threshold, to the dispenser, one or more instructions to modify an amount of substance dispensed per usage event by the dispenser 200. For instance, if there is a low level of the substance 204 remaining in the dispenser 200, then the instructions can cause the dispenser to reduce the amount of substance 204 dispensed per usage event until the service agents refill the dispenser 200 with the substance 204.

In some embodiments, the dispenser manager 410 is configured to determine the threshold based on at least one of location data corresponding to a location of the dispenser or usage history data corresponding to previous usage, dispensing or service events of the dispenser. The usage history data can indicate a service time. The service time can indicate an amount of time between a first time during which a service request is generated and a second time during which the service is performed. The dispenser manager 410 can determine a threshold service time based on acceptable amount of time between generating the service request and performing the service. The threshold service time can differentiate between quickly serviced dispensers and dispensers requiring more service time. The dispenser manager 410 can determine a substance remaining threshold based on the service time and the threshold service time. For instance, dispensers with service times exceeding the threshold service time can have high substance remaining thresholds. The high substance remaining thresholds can cause the dispenser manager 410 to determine that the dispenser 200 can have the substance refill despite the dispenser 200 having a medium amount of the remaining substance. Similarly, dispensers with service times not exceeding the service threshold can have low substance remaining thresholds. The low substance remaining thresholds that cause the dispenser manager 410 to determine that the dispenser 200 needs to have the substance refilled only when the dispenser 200 has a low amount of the substance 204 remaining.

The dispenser manager 410 can also generate a service request. The dispenser manager 410 can generate the service request responsive to the dispenser manager 410 determining that the dispenser 200 needs a substance refill. The service request can indicate that the dispenser 200 needs service. The service request can be a notification that indicates that the dispenser 200 needs service. For instance, if the substance remaining in the dispenser 200 satisfies a threshold, then the dispenser manager 410 can transmit the service request to an agent selected to perform the service. The service request can be the refill request.

The dispenser manager 410 can determine that the dispenser 200 needs service. Responsive to determining that the dispenser 200 needs service, the dispenser manager 410 can generate a service request. The dispenser manager 410 can transmit the service request to the agent. In some embodiments, the dispenser manager 410 generates the service request responsive to receiving the request for service from the dispenser 200. The service request can indicate that agents can refill the dispenser 200 with substance 204. The service request can include a priority set by a service priority policy. The priority can indicate how the agent manager 412 should allocate service resources to the dispenser 200. For instance, if the dispenser manager 410 determines that the substance level in the dispenser 200 is low but not empty, the dispenser manager 410 can assign a medium service priority to the service request. Similarly, the dispenser manager 410 can assign a higher service priority to a broken dispenser than an operational dispenser having a low level of substance remaining.

In some embodiments, the dispenser manager 410 selects, from a plurality of agents, the agent to which to provide the refill request or the service request. The dispenser manager 410 can select the agent based on a current time, a location of the agent, or service capabilities of the agent. The dispenser manager 410 can select the agent based on the service priority level of the service request. For instance, the dispenser manager 410 can assign available agents to service requests with a high service priority, or the dispenser manager 410 can assign busy agents to service requests with a low service priority.

The dispenser manager 410 can forward, via the communications module 404, the service request from the dispenser manager 410 to the agent. The dispenser manager 410 can forward, via the communications module 404, the service request to the agent selected by the agent manager 412. The communications module 404 can communicate with the agents via at least one of a LAN connector, a Bluetooth connector, an RFID connector, or a cellular connector. The communications module 404 can send the service requests to the agents indicating that the dispenser needs service or refill. The dispenser manager 410 can send service requests to the agents as an email, a cell phone notification, or a push notification to a service app. The service request can include dispenser 200 information such as location, what service is required, the substance 204 required, and a quantity of the substance 204 needed.

The dispenser manager 410 can also manage the power consumption of the dispenser 200. In some embodiments, the dispenser manager 410 provides the service request to an agent based on the power consumption of the dispenser 200. For instance, if the dispenser 200 is not consuming any power, then the dispenser 200 is in a power off state. The dispenser manager 410 can identify that the dispenser 200 is a power off state. Responsive to identifying that the dispenser manager 410 is a power off state, the dispenser manager 410 can generate a service request and transmit the service request to the agent. The service request can indicate that the dispenser 200 requires service. The dispenser manager 410 can also determine whether the power consumption exceeds the amount of energy provided. For instance, if the dispenser manager 410 determines that the power consumption exceeds the amount of energy provided, then the dispenser manager 410 can determine that the energy provider 232 of the dispenser 200 cannot provide enough energy for the dispenser 410. Responsive to the energy provider 232 failing to provide enough energy to the dispenser 200, the dispenser manager 410 can notify the agents. Responsive to receiving service requests, the agents can service the dispensers. The agents can service the dispensers by changing the batteries, fixing the substance dispensers, refilling substance, or by fixing any other dispenser issue.

The dispenser manager 410 can adjust the power consumption of the dispenser 200. The dispenser manager 410 can assign the dispenser 410 to a low power mode. During the low power mode, the dispenser manager 410 can generate instructions to alter the power consumption of the dispenser 200. The instructions can indicate that the dispenser 200 avoid using energy for the substance dispensing, and instead use energy to provide decontamination services with the light 206. Providing decontamination services with the light 206 can increase the number of actuation events.

In some embodiments, the dispenser manager 410 transmits, responsive to determining that the amount of substance 204 remaining in the dispenser satisfies the threshold, one or more instructions to modify a length of time, number of light actuation events, or an intensity of light emitted by the dispenser 200. For instance, if the dispenser 200 has a low substance levels, then the instructions can indicate that the dispenser 200 can dispense less substance, and instead provide decontamination services with the light 206.

The dispenser manager 410 can further determine usage trends to track the usage of the dispenser 200 over time. The dispenser manager 410 can determine that the dispenser 200 needs service based on changes in usage trends. For instance, a decrease in the number of substance dispensing events can indicate that the dispenser cannot detect users, or an increase in the number of substance dispensing events per user can indicate that the substance dispenser is malfunctioning. In some embodiments, the dispenser manager 410 determines, for the dispenser 200, a first usage trend based on a first plurality of usage events received from the dispenser during a first time window and a second usage trend based on a second plurality of usage events received from the dispenser during a second time window. The first plurality of usage events can be substance-dispensing events over a first time, and the first usage trend can indicate a first number of substance dispensing events per unit of time during the first time. The second plurality of usage events can be substance-dispensing events over a second time, and the second usage trend can indicate a second number of substance dispensing events per unit of time during the second time. The dispenser manager 410 can compare the first usage trend to the second usage trend. For instance, the dispenser manager 410 can compare a first number of usage events per unit time to the second number of usage events per unit time. Responsive to determining that the second usage trend is different from the first usage trend, the dispenser manager 410 can be configured to adjust a service schedule according to which service is provided to the dispensers, adjust a refill schedule for refilling substance in the dispensers, or transmit a service request to the agent. For instance, if the dispenser manager 410 determines that the dispenser 200 is using more substance 204 over the second usage trend compared to the first usage trend despite no other changes such as number of dispensing events, then the dispenser manager 410 can determine that the substance dispenser has a substance leak. Responsive to determining the substance leak, the dispenser manager 410 can generate the service request.

To determine when to service the dispenser 200, the dispenser manager 410 can estimate a dispensing rate (e.g., amount of substance dispensed on a daily basis) based on history of recorded dispensing events and/or information indicative of substance amount dispensed per event. The estimate dispensing rate can be computed as an average of amount of substance 204 dispensed (e.g., per day) for the last n days where n is an integer. The dispenser manager 410 can obtain and store information indicative of the amount of substance 204 dispensed at each dispensing event. The dispenser manager 410 can use the estimated dispensing rate and information indicative of substance dispenser 202 or container 310 capacity to predict a potential supply/refill date. For instance, the dispenser manager 410 can divide the capacity of the container 310 (or cumulative capacity of all containers in the substance dispenser 202) by the estimated dispensing rate to calculate a duration (e.g., number of days) to consume the substance 204. The dispenser manager 410 can set the supply/refill date few days (e.g., a week, five days, or other period) prior to complete consumption of substance in the container 310 or in a plurality of containers.

The dispenser manager 410 can also remotely manage the inventory of the substance 204 for one or more dispensers. The dispenser manager 410 can offer substance inventory management to service providers as a service. For instance, a service option can include full inventory management by the dispenser manager 410, in which case, the dispenser manager 410 tracks the inventory of the substance 204 (and/or dispensers). By tracking substance levels among the dispensers and agents, the dispenser manager 410 can detect when the dispensers or the agents have low substance levels. The low substance levels can indicate that the dispensers do not have enough substance to fulfill substance-dispensing requests or that the agents do not have enough substance to fulfill substance refills. The dispenser manager 410 can automatically generate substance replenishment requests. The substance replenishment requests can include information about the substance type and amount for the dispensers. The dispenser manager 410 can generate substance replenishment requests responsive to detecting low levels of substance among the dispensers or among the agents providing substance refills. Agents (or other employees) associated with the service provider can approve such substance replenishment requests before the dispenser manager 410 sends them to the substance 204 and/or dispenser 200 suppliers. By automatically ordering supplies for the dispensers and agents, the full inventory management by the dispenser manager 410 can result in reduced costs for service providers opting for such service option.

In some embodiments, the dispenser manager 410 can determine a refill date of the substance 204 or a substance refill request. In response, the dispenser manager 410 can check a substance refill date stored in the dispenser profile 416 of the dispenser 200 (or compare the received date to a respective date stored in the dispenser profile 416 of the dispenser 200). The dispenser manager 410 can then determine a refill event based on the date checking (or date comparing).

The dispenser manager 410 can generate a dashboard. The dashboard can display the usage data for each dispenser 200 managed by the dispenser manager 410. The service agents can access the dashboard. The dashboard can display, for each dispenser 200, a substance capacity, a substance threshold, and amount of substance remaining. The dashboard can display the usage a status of the dispensers. The status can indicate whether the dispenser 200 is operational or whether the dispenser 200 needs service. The dashboard can display different colors for each status and each dispenser 200. For example, the dashboard can highlight dispensers that need service on a map of dispensers. The dashboard can display the usage data in a time series format. For instance, the dashboard can display the dispensing of substance 204 over time, energy usage over time, and service over time. The dispenser manager 410 can determine usage patterns from the time series data. Based on the usage pattern, the dashboard can determine or predict supply or substance 204 reordering. The dashboard can allow a service agent to order the substance 204 or the supplies for the dispensers.

The agent manager 412 can include hardware, software or a combination of hardware and software. The agent manager 412 can include a script, program, file, or other software construct, which when executed by the processor 402, can manage the data associated with each agent. The agent manager 412 can maintain, in the one or more data structures, agent profiles for the agents. Each agent profile 418 can store the data corresponding to each agent. The agents can provide various services to the dispensers, such as refilling of the substance 204 or fixing the light 206. The agent profile 418 can include a location of agent, service capabilities of agent, or substance refill availability.

Actions performed by the dispenser manager 410 to initiate the service process, such as which service agent to request or what substance 204 they need to refill the dispenser 200 with, can differ based on the determination of the dispenser manager 410 and the data available for each agent in the agent profile 418. For instance, the dispenser manager 410 can determine that the dispenser 200 requires refill with a particular substance, and the dispenser manager 410 can determine the agent that can refill the dispenser with the particular substance. The dispenser manager 410 can also determine that the agent is closest to the dispenser 200 or is familiar with servicing the dispenser 200.

In some embodiments, the agent manager 412 generates access data. The access data can allow the agents to service the dispenser 200. The agent manager 412 can transmit, to the dispenser 200 or a communication device of the agent, access data configured to unlock the dispenser. The access data can unlock a door of the dispenser. The agents can request the access data to service the dispenser. Without the access data, the dispenser manager 410 can identify attempts to service the dispenser as tampering. The usage event tracker 230, as previously discussed, can receive the tampering indication.

The user manager 414 can include hardware, software or a combination of hardware and software. The user manager 414 can include a script, program, file, or other software construct, which when executed by the processor 402, can manage the data associated with each user of the dispensers. The user manager 414 can maintain, in the one or more data structures, user profiles for the users. Each user profile 420 can include the usage data corresponding to the use of the dispenser by the user corresponding to the user profile. Each user profile 420 can store the data corresponding to each user.

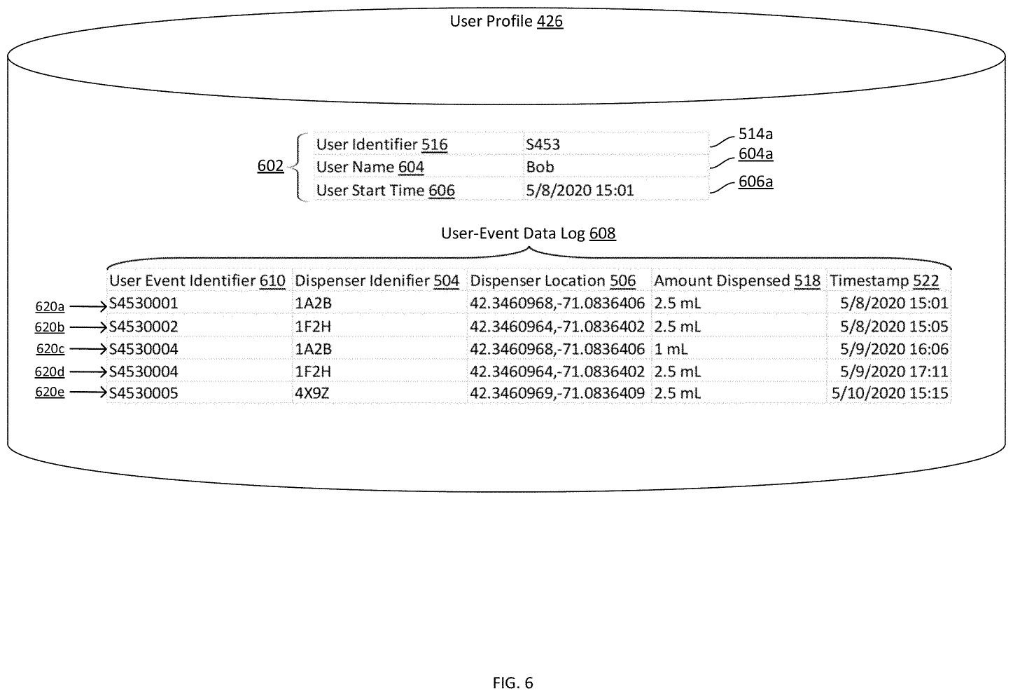

Now referring also to FIG. 6, depicted are exemplary data attributes of the user profile 420. The user profile 420 can include specification user data 602. The specification user data 602 can be preset or static data corresponding to the dispenser. The specification user data 602 can include the user identifier 516, a user name 604 indicating the name of the user, and a user start time 606 indicating a time of discovery of the user by the multi-dispenser management system 120. The user profile 420 can include user-event data log 608. The user-event data log 608 can include one or more user entries 620a-e. Each user entry 620 can include a user event identifier 610 identifying each interaction of the user with the dispensers, the dispenser identifier 504, the dispenser location 506, the amount dispensed 518, and the timestamp 522. In some embodiments, the user profile 420 can receive and store additional data attributes from the dispenser manager 410, such as light settings and light usage of the dispenser 200. For instance, the dispenser event identifier 514 can correspond to a data structure storing that the light was on during the dispensing event, or any other parameter transmitted by the light controller 224 of the dispenser 200 and received by the usage event receiver 406 for analysis by the dispenser manager 410. The data can be stored in one or more data structures. The data can be in hexadecimal, numerical, or any other database format.

In an exemplary embodiment, user name 604a of the dispenser 200 is “Bob.” User start time 606a can indicate that the multi-dispenser management system 120 detected “Bob” on “5/8/2020” and at “15:01”. User event identifiers 610a-610e correspond to activity of the user. Each user entry 620 stored in the user event data log 608 can correspond to a user interaction with any of the dispensers. Each user entry 620 includes a user event identifier uniquely identifying the event, a dispenser identifier uniquely identifying the dispenser, a dispenser location, an amount of substance dispensed, and a timestamp indicating a time at which the event occurred. The dispenser identifier can indicate the specific dispenser that interacted with the user. The dispenser location indicates the location of the specific dispenser during the user interaction. The dispenser location can indicate the location of the user during the user interaction. The amount dispensed indicates how much of the substance 204 the user received. The timestamp indicates when the user interaction with the dispenser 200 took place. Each user event identifier 610 can also correspond to light settings and light usage corresponding to the dispenser 200. For instance, the user event identifier 610 can correspond to a data structure storing that the light was on during the dispensing event, or any other parameter transmitted by the light controller 224 of the dispenser 200 and received by the usage event receiver 406 for analysis by the dispenser manager 410.

The user manager 414 can customize the experience of each user with the dispensers based on the data in the corresponding user profile 420. The user manager 414 can generate a custom dispensing signal for each user. The custom dispensing signal can indicate the amount of the substance dispensing requested. The user manager 414 can generate the custom dispensing signal based on user preferences, user size, or user history. The dispenser manager 410 or the user manager 414 can transmit the custom dispensing signal to the dispenser 200. For instance, upon receiving the custom dispensing signal, the dispenser 200 can provide a user with larger hands a greater amount of substance 204. Alternatively, upon receiving another custom dispensing signal, the dispenser 200 can reduce the amount of the substance 204 dispensed. Responsive to the user manager 414 determining that the usage history of the user exceeds a usage threshold, the dispenser manager 410 or the user manager 414 can generate a custom dispensing signal indicating that the user receive less substance from the dispenser 200. For instance, the custom dispensing signal can indicate that the user whose use of the substance 204 exceeds the usage threshold cannot dispense the substance 204. The dispenser manager 410 can define the usage threshold for each dispenser 200. The user manager 414 can also share the user profile 420 with the dispensers. In some embodiments, the user manager 414 shares user profiles with dispensers corresponding to dispenser identifiers stored in the user-event data log 608. For instance, the user manager 414 can share, with dispensers that a user has previously interacted with, the user's user profile.

As described above in FIG. 2, the user detector 228 can detect users of the dispensers. In some embodiments, the user detector 228 can use the data of the user profiles or the custom dispensing signals to modify the behavior of the dispenser 200. For instance, the user detector 228 can use the data to determine a dispensing policy according to which dispense the substance 204, stop tracking particular users, maintain use count for users, or block users after a certain number of uses. For instance, the dispensing policy can indicate which users prefer to receive less substance from the substance dispenser. The data of the user profiles or the custom dispensing signals can also indicate which users the dispensers cannot track, or the data of which users the dispensers cannot include in the dispenser-event data log 512 or the user-event data log 608. Similarly, the light controller 224 can modify the parameters of the light 206 based on the data of the user profiles or the custom dispensing signals. For instance, the data of the user profiles or the custom dispensing signals can indicate that certain users do not want to use the light, while other users want the light to shine at maximum brightness. The light controller 224 can modify the parameters of the light 206 based on the data of the user profiles or the custom dispensing signals. For instance, the light controller 224 can control the light 206 to stay off for certain users and shine at maximum brightness for other users.

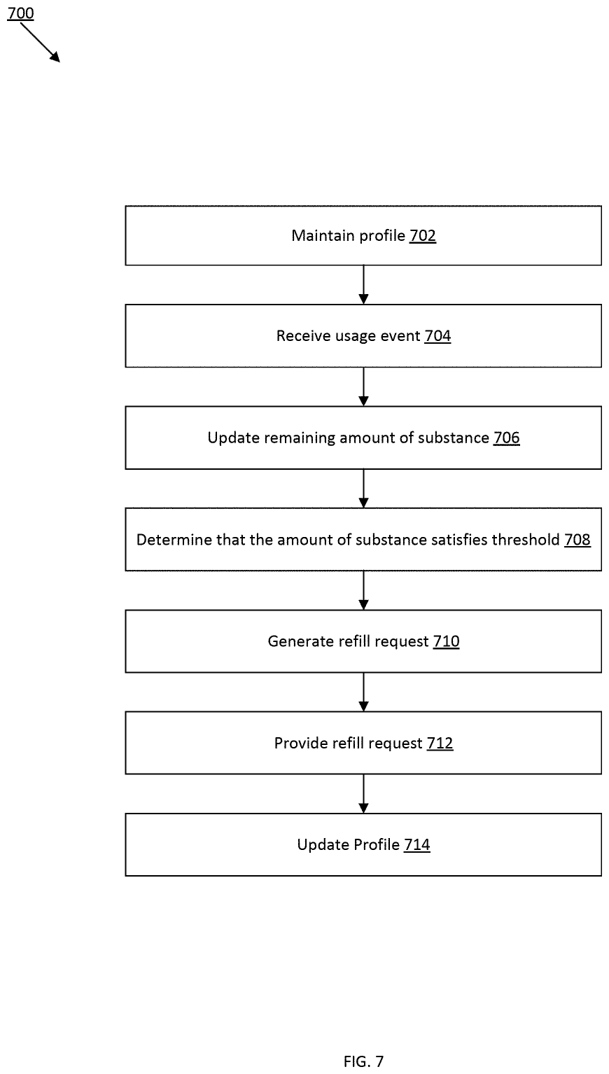

A method 700 for managing a plurality of dispensers. Now referring to FIG. 7, the method 700 can include maintaining a profile (step 702). The method 700 can further include receiving a usage event (step 704). The method 700 can further include updating a remaining amount of substance (step 706). The method 700 can further include determining that the amount of substance satisfies a threshold (step 708). The method 700 can further include generating a refill request (step 710). The method 700 can further include providing the refill request (step 712). The method 700 can further include updating the profile (step 714).

In further detail, the multi-dispenser management system 120 can perform the method 700. The method 700 can include maintaining a profile (step 702). A system such as the multi-dispenser management system 120 can maintain one or more profiles. The profiles can include the dispenser profile 416, the agent profile 418, and the user profile 420. The multi-dispenser management system 120 maintains the profiles via the dispenser manager 410, the agent manager 412, and the user manager 414. Each manager can assign the data to a particular dispenser, agent, and user. The managers can add or update data entries to maintain a data log. The managers can manage static and dynamic information. The managers can update data in the profiles. Profiles are stored on one or more data structures or network elements accessible to the multi-dispenser management system 120.

In some embodiments, the multi-dispenser management system 120 can maintain the dispenser profile 416 shown in FIG. 5. The dispenser profile 416 can include static data, preset data, and a dispenser-event data log corresponding to each dispenser 200. The dispenser profile can include dispenser specific attributes and parameters to assist the multi-dispenser management system in optimizing the availability of the dispensers. For instance, the dispenser profile can indicate which dispensers have low levels of substance or need service, so the multi-dispenser management system 120 can generate service requests to order supplies for those dispensers and have those dispensers serviced.

In some embodiments, the multi-dispenser management system 120 can maintain the agent profile 418. The agent profile 418 can include a location of agent, service capabilities of agent, or substance refill options. The data can include agent specific attributes and parameters to assist the multi-dispenser management system 120 in selecting agents to service the dispensers. For instance, the multi-dispenser management system 120 can determine and maintain the location of service agents and their service capabilities.

In some embodiments, the multi-dispenser management system 120 can maintain the user profile 420 shown in FIG. 6. The user profile 420 can include static data, preset data, and a user-event data log corresponding to each user. The data can include user specific attributes and parameters to assist the multi-dispenser management system 120 in customizing the operation of the dispensers based on user preferences. For instance, the data of the user profiles can indicate that certain users do not want to any substance dispensed, while other users want a maximum amount of substance.

The method 700 can include receiving a usage event (step 704). The multi-dispenser management system 120 can receive the usage event from the dispenser 200. The multi-dispenser management system 120 can process the received data via the usage event receiver 406 and the substance amount monitor 408. The data can include data packets corresponding to the usage event. The usage event can include the data parameters described in FIGS. 5 and 6. The data can identify the remaining amount of substance in the device.

The method 700 can include updating a remaining amount of substance (step 706). The multi-dispenser management system 120 can process the update via the dispenser manager 410. The multi-dispenser management system 120 can update the dispenser profiles of the dispensers. The multi-dispenser management system 120 can update the remaining amount of the substance 204 by replacing older data of remaining amount of substance with newer data of the amount of the substance 204. The multi-dispenser management system 120 can determine the amount of substance remaining from a dispensed amount. The multi-dispenser management system 120 can subtract the dispensed amount from the previous remaining amount of the substance 204. The multi-dispenser management system 120 can store the amount of substance remaining by updating the corresponding profile value of the corresponding dispenser profile with the amount of the substance remaining. The multi-dispenser management system 120 can match the remaining balance of the substance 204 or the amount of the substance 204 dispensed by the dispenser to a corresponding usage event. In some embodiments, the multi-dispenser management system 120 can update user profiles to indicate usage events. The multi-dispenser management system 120 can update user profiles to indicate usage events by adding data entries indicating the usage events.

The method 700 can include determining that the amount of substance satisfies a threshold (step 708). Upon determining the remaining amount of the substance 204, the multi-dispenser management system 120 can compare the remaining amount to a substance threshold. The multi-dispenser management system 120 can set the substance threshold based on amount of time to refill device, previous usage, or a preset value. Based on the comparison, the multi-dispenser management system 120 can identify that the remaining amount of substances satisfies the substance threshold.

The method 700 can include generating a refill request (step 710). The multi-dispenser management system 120 can generate the refill request. The refill request can indicate that the dispenser 200 needs service. The refill request can indicate that the dispenser 200 needs a substance refill. The multi-dispenser management system 120 can generate the refill request responsive to the multi-dispenser management system 120 determining that the dispenser 200 can receive the refill of substance 204. For instance, if the amount of the substance 204 remaining in the dispenser 200 satisfies the substance threshold, then the multi-dispenser management system 120 can generate the refill request.

The refill request can also be a service request. The service request can indicate that the particular dispenser needs service by agent. The service can be for any part of the dispenser 200, such as the stand 302, the light 206, or the energy provider 232. The multi-dispenser management system 120 can generate the service request responsive to the usage data satisfying an error threshold. For instance, if the usage data satisfies the error threshold, then the multi-dispenser management system 120 can generate the service request to the agent manager 412. The error threshold can be a power level, light voltage, a substance amount, or any other type of data corresponding to the dispensers. For instance, if the light voltage is below the error threshold for the light, that the service request can be generated.

The service request can include a priority set by a service priority policy. The priority can indicate how the agent manager 412 should allocate service resources to the dispenser 200. For instance, if the dispenser manager 410 determines that the substance 204 level in the dispenser 200 is low but not empty, the multi-dispenser management system 120 can assign a medium service priority to the service request. Similarly, the multi-dispenser management system 120 can assign a higher service priority to a broken dispenser than an operational dispenser having a low level of substance.

The method 700 can include providing the refill request (step 712). The multi-dispenser management system 120 can transmit the refill request or the service request. The multi-dispenser management system 120 can transmit the service request or the refill request to the agent manager 412. The multi-dispenser management system 120 can select, from a plurality of agents, the agent to which to provide the refill request or fulfill the service request. The multi-dispenser management system 120 can select the agent based on a current time, location of the agent, or service capabilities of the agent. For instance, the multi-dispenser management system 120 can select service agents to service dispensers with a nearby location. The multi-dispenser management system 120 can also select based on the priority level of the service request. For instance, the multi-dispenser management system 120 can assign available agents to service requests with a high priority, while assigning busy agents to service requests with a low priority. The multi-dispenser management system 120 can transmit the refill requests or the service request to the agents. The multi-dispenser management system 120 can transmit the refill requests or the service request to the agents via the network 104. The multi-dispenser management system 120 can transmit the refill request or the service request to the selected agent.

The method 700 can include updating the profile (step 714). The multi-dispenser management system 120 can update the dispenser profile with the remaining amount of the substance 204. The multi-dispenser management system 120 can update the dispenser profile with the remaining amount of the substance 204 responsive to the agents adding additional substance to the dispenser 200. The multi-dispenser management system 120 can update the dispenser profile with information about the service performed by the agents to the dispenser.

The multi-dispenser management system 120 can update the agent profiles of agents that receive or fulfill the service requests or refill requests. The multi-dispenser management system 120 can update the agent profiles with the type of services performed by the agent or the number of times the agent performed each type of service on the dispensers. For instance, the update to the agent profile can indicate that the agent refilled the substance of dispensers located in a first terminal of an airport, and has refilled the substance of those dispenser eight times.

While the invention has been particularly shown and described with reference to specific embodiments, it should be understood by those skilled in the art that various changes in form and detail may be made therein without departing from the spirit and scope of the invention described in this disclosure.

While this specification contains many specific embodiment details, these should not be construed as limitations on the scope of any inventions or of what may be claimed, but rather as descriptions of features specific to particular embodiments of particular inventions. Certain features described in this specification in the context of separate embodiments can also be implemented in combination in a single embodiment. Conversely, various features described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

1. A dispenser system comprising:
a housing including a compartment for a container containing a substance to be dispensed;
a substance dispenser configured to dispense the substance from the container, the substance dispenser coupled to a substance-monitoring sensor;
a light emitter configured to emit light;
an energy provider configured to provide energy to the substance dispenser and the light emitter;
a communications module configured to communicate data between the dispensing system and a server; and
one or more processors configured to:
receive a request to initiate a sanitation process;
actuate the substance dispenser to dispense the substance responsive to the request;
actuate the light emitter to emit light;
determine, from the substance monitoring sensor, an amount of substance remaining in the container; and
transmit, via the communications module, the amount to the server.

2. The dispenser system of claim 1 comprising a door coupled to the housing, the door comprising a lock configured to restrict access to the container.

3. The dispenser system of claim 1 comprising a user detector configured to detect a presence of a user, wherein receiving the request to initiate the sanitation request comprises generating the request responsive to the user detector detecting the presence of the user.

4. The dispenser system of claim 3 wherein the user detector is configured to identify the user and wherein the one or more processors are configured to transmit, via the communications module to the server, usage data identifying the user subsequent to the user detector identifying the user.

5. The dispenser system of claim 1 wherein the housing includes a mount configured to mount the housing to a wall surface or a stand.

6. The dispenser system of claim 1 wherein the energy provider includes a magnetic levitation system configured to generate energy to control the dispenser, the magnetic levitation system including a first magnet and a second magnet, the first magnet configured to exert a magnetic force on the second magnet responsive to the actuation of the substance dispenser to cause the magnetic levitation system to generate energy for a subsequent actuation of the substance dispenser.

7. The dispenser system of claim 1 wherein the substance monitoring sensor is configured to determine the amount of the substance remaining in the container based on at least one of a weight of the substance remaining in the container, a visual marker indicating the amount of substance remaining in the container, or a force applied by the container to the substance monitoring sensor.

8. The dispenser system of claim 1 further comprising an actuator, the actuator actuated by the user to generate energy to be used by the energy provider.

9. The dispenser system of claim 1 wherein the energy provider includes at least one of a solar panel, a wind turbine, or a battery.

10. A multi-dispenser management system comprising:
a communications manager configured to communicate with a plurality of dispensers; and
one or more processors coupled to memory and configured to:
maintain, for each dispenser of the plurality of dispensers, in one or more data structures, a device profile including an amount of a substance remaining in the dispenser and location data of the dispenser;
receive, from a dispenser, usage data corresponding to an usage event at the dispenser, the usage data identifying the dispenser;
update, based on the usage data, the amount of substance remaining in the dispenser;
determine that the amount of substance remaining in the dispenser satisfies a threshold corresponding to the dispenser;
generate, responsive to determining that the amount of substance remaining in the dispenser satisfies the threshold, a refill request to refill the dispenser with the substance, the refill request identifying the dispenser; and
provide the refill request to an entity.

11. The multi-dispenser management system of claim 10 wherein the one or more processors are configured to identify from the usage data, a user corresponding to the usage data, and maintain, in the one or more data structures, a user profile for the user including the usage data.

12. The multi-dispenser management system of claim 10 wherein the usage data includes a quantity of substance dispensed by the dispenser, and wherein the one or more processors are configured to update the amount of substance remaining in the dispenser based on the quantity of substance dispensed.

13. The multi-dispenser management system of claim 10, wherein the one or more processors are configured to determine the threshold based on at least one of location data corresponding to a location of the dispenser or usage history data corresponding to previous usage events of the dispenser.

14. The multi-dispenser management system of claim 10 wherein the one or more processors are configured to determine a timestamp corresponding to the usage data received from the dispenser and determine the threshold based on the timestamp.

15. The multi-dispenser management system of claim 10, wherein the one or more processors are configured to select, from a plurality of entities, the entity to which to provide the refill request, the selection based on a current time or a location of the entity.

16. The multi-dispenser management system of claim 10, wherein the usage data includes power data corresponding to a current power state of the dispenser, the one or more processors are configured to provide a notification to an entity based on the current power state.

17. The multi-dispenser management system of claim 10, wherein the one or more processors are configured to:
determine, for the dispenser, a first usage trend based on a first plurality of usage events received from the dispenser during a first time window and a second usage trend based on a second plurality of usage events received from the dispenser during a second time window; and
transmit a notification to an entity responsive to determining that the second usage trend is different from the first usage trend.

18. The multi-dispenser management system of claim 10, wherein the one or more processors are configured to transmit, to a client device of the entity, door access data configured to unlock a door of the dispenser.

19. The multi-dispenser management system of claim 10, wherein the one or more processors are configured to transmit, responsive to determining that the amount of substance remaining in the dispenser satisfies the threshold, one or more instructions to modify an amount of substance dispensed per usage event.

20. The multi-dispenser management system of claim 10, wherein the one or more processors are configured to transmit, responsive to determining that the amount of substance remaining in the dispenser satisfies the threshold, one or more instructions to modify a length of time or an intensity of light emitted by the dispenser.
